(19) United States
(12) Patent Application Publication
(10) Pub. No.: US 2019/0069501 A1
(43) Pub. Date: Mar. 7, 2019

(54) LETTUCE VARIETY NUN 07839 LTL

(71) Applicant: NUNHEMS B.V., AB Nunhems (NL)

(72) Inventor: Johan Engelbert van Zee, Zaltbommel (NL)

(73) Assignee: NUNHEMS B.V., AB Nunhems (NL)

(21) Appl. No.: 15/903,573

(22) Filed: Feb. 23, 2018

Related U.S. Application Data
(60) Provisional application No. 62/555,079, filed on Sep. 7, 2017.

(51) Int. Cl. A01H 5/12; C12N 5/04; A01H 4/00; A01H 1/02

(52) U.S. Cl. CPC A01H 5/12; A01H 1/02; A01H 4/008; C12N 5/04

(57) ABSTRACT

The invention relates to a new variety of lettuce designated NUN 07839 LTL as well as seeds and plants and heads or leaves thereof.

### CROSS REFERENCE TO RELATED APPLICATIONS

This application claims the benefit of U.S. Provisional Application Ser. No. 62/555,079, filed Sep. 7, 2017, the contents of which are herein incorporated by reference in its entirety.

### FIELD OF THE INVENTION

The present invention relates to the field of plant breeding and, more specifically, to the development of NUN 07839 LTL (also designated as NUN 07839 or 07839 LTL or NUN 07839 or 07839 LTL. The invention further relates to vegetative reproductions of NUN 07839 LTL, methods for in vitro tissue culture of NUN 07839 LTL, an explant and also to phenotypic variants of NUN 07839 LTL.

The goal of vegetable breeding is to combine various desirable traits in a single variety. Such desirable traits may include greater yield, resistance to diseases, insects or other pests, tolerance to heat and drought, better agronomic quality, higher nutritional value, enhanced growth rate and improved shelf life.

The development of commercial lettuce cultivars or varieties requires the crossing of lettuce plants, and the evaluation of the crosses. Pedigree breeding and recurrent selection are examples of breeding methods are used to develop cultivars from breeding populations. Breeding programs combine desirable traits from two or more varieties or various broad-based sources into breeding pools from which cultivars are developed by selfing and selection of desired phenotypes. The new cultivars are crossed with other varieties and the inbred lines or hybrids from these crosses are evaluated to determine which have commercial potential.

All cultivated forms of lettuce belong to the highly polymorphic species Lactuca sativa that is grown for its edible head and leaves. Lactuca sativa is in the Asteraceae (Compositae) family Lettuce is related to chicory, sunflower, aster, dandelion, artichoke and chrysanthemum. L. sativa is one of about 300 species in the genus Lactuca. There are many types of lettuce, and new types are constantly in development. Types of lettuce include Cutting/Leaf, Iceberg/Crisphead, Cos or Romaine, Batavian, Salinas Group, Latin, Butterhead, Great Lakes Group, Eastern (Ithaca) Group, Bibb, Vanguard Group, multileaf or Stem lettuce.

Fresh lettuce is available in the United States year-round although the greatest supply is from May through October. For planting purposes, the lettuce season is typically divided into three categories, early, mid and late, with the coastal areas planting from January to August, and the desert regions planting from August to December. Lettuce is consumed nearly exclusively as fresh, raw product and occasionally as a cooked vegetable.

Lifestyles change and the demand from restaurants and catering firms for colorful and interesting garnish for sandwiches and ready-to-use processed salads continue to rise. As a result, there is a demand for breeding companies to develop new varieties with specific shapes of leaves, specific average size of leaves, glossiness, prominent color and a wide variety of texture, as well as good yield.

### SUMMARY OF THE INVENTION

In an aspect of the invention, a seed of lettuce variety NUN 07839 LTL is provided, wherein a representative sample of said seed has been deposited under Accession Number NCIMB 42969. The seed of the lettuce variety of the invention may be provided as an essentially homogeneous population of lettuce seed. Therefore, seed of the invention may be defined as forming at least about 97% of the total seed, including at least about 98%, 99% or more of the seed. The population of lettuce seed may be particularly defined as being essentially free from other seed. The seed population may be grown to provide an essentially homogeneous population of lettuce plants according to the invention. Also encompassed are a plant grown from a seed of lettuce variety NUN 07839 LTL and a plant part of said variety.

In another aspect the invention provides for a variety of Lactuca sativa called NUN 07839 LTL. The invention also provides for a seed or a plurality of seeds of said new variety, plants produced from growing the seed of the new variety NUN 07839 LTL, and progeny of any of these. Especially, a plant or a progeny retaining all or all but one, two or three of the “distinguishing characteristics” or all or all but one, two or three of the “morphological and physiological characteristics” NUN 07839 LTL referred to herein, is encompassed herein as well as methods for producing these.

In one aspect, such a plant or such a progeny has all the physiological and morphological characteristics of variety NUN 07839 LTL when grown under the same environmental conditions. In another aspect such progeny have all the physiological and morphological characteristics as listed in Table 1 and/or 2 for NUN 07839 LTL when measured under the same environmental conditions (i.e. evaluated at significance levels of 1%, 5% or 10% significance, which can also be expressed as a p value).

In another aspect a plant of NUN 07839 LTL or said progeny plants has 7, 8, or more or all of the distinguishing characteristics: 1) ______; 2) ______; 3) ______; 4) ______; 5) ______; 6) ______; 7) ______; 8) ______; 9 ______: and 10) ______. NUN 07839 LTL is a red oak babyleaf lettuce.

Further, a lettuce head and/or a lettuce leaf produced on a plant grown from these seeds is provided.

In yet another embodiment of the invention, a plant having one, two or three physiological and/or morphological characteristics which are different from those of the lettuce variety of the invention and which otherwise has all the physiological and morphological characteristics of NUN 07839 LTL as listed in Table 1 and/or 2, wherein a representative sample of seed of variety NUN 07839 LTL has been deposited under Accession Number NCIMB 42969.

Further, a vegetatively propagated plant of variety NUN 07839 LTL, or a part thereof, is provided having all or all but one, two or three of the morphological and physiological characteristics NUN 07839 LTL when grown under the same environmental conditions.

Also a plant part obtained from variety NUN 07839 LTL is provided, wherein said plant part is selected from the group consisting of: a fruit, a part of a fruit, a leaf, a part of a leaf, a head, a part of a head, pollen, an ovule, a cell, a petiole, a shoot or a part thereof, a stem or a part thereof, a root or a part thereof, a root tip, a cutting, a seed, a part of a seed, seedcoat or another maternal tissue which is part of a seed grown on said variety, hypocotyl, cotyledon, a scion, a stock, a rootstock, a pistil, an anther, and a flower or a part thereof. Heads and leaves are particularly important plant parts. In yet another aspect, a seed of NUN 07839 LTL is provided. In still another aspect, a seed growing or grown on a plant of NUN 07839 LTL is provided.

In yet another aspect, a seed of NUN 07839 LTL is provided (i.e. a seed which when grown grows into variety NUN 07839 LTL). In still another aspect, a seed growing or grown on a plant of NUN 07839 LTL is provided (i.e. after pollination of the flower of NUN 07839 LTL).

### DEFINITIONS

All patent and non-patent literatures cited herein are incorporated by reference in their entireties.

“Lettuce” refers herein to plants of the species Lactuca sativa L. The most commonly eaten parts of a lettuce plant are the head or a leaf. The head comprises a core and leaves, which may be divided in inner and outer leaves.

“Cultivated lettuce” refers to plants of Lactuca sativa i.e. varieties, breeding lines or cultivars of the species L. sativa as well as crossbreds thereof, or crossbreds with other Lactuca sativa species, or even with other Lactuca species, cultivated by humans and having good agronomic characteristics; preferably such plants are not “wild plants”, i.e. plants which generally have much poorer yields and poorer agronomic characteristics than cultivated plants and e.g. grow naturally in wild populations. “Wild plants” include for example ecotypes, PI (Plant Introduction) lines, landraces or wild accessions or wild relatives of Lactuca sativa, comprising, for example L. virosa or L. serriola, and other related species.

The terms “NUN 07839 LTL”, “NUN 7839 LTL”, “lettuce NUN 07839 LTL”, “NUN 07839”, “07839 LTL” or “variety NUN 07839 LTL” are used interchangeably herein and refer to a lettuce plant of variety NUN 07839 LTL, representative seed of which having been deposited under Accession Number NCIMB 42969.

A “seed of NUN 07839 LTL” refers to a seed of the inbred variety NUN 07839 LTL represented by the deposit with Accession Number NCIMB 42969. It can be grown into a plant of NUN 07839 LTL.

An “embryo of NUN 07839 LTL” refers to an embryo as present in a seed of the inbred variety NUN 07839 LTL, a representative sample of said seed of NUN 07839 LTL having been deposited under Accession Number NCIMB 42969.

A “seed grown on NUN 07839 LTL” refers to a seed grown on a mature plant of NUN 07839 LTL or inside a fruit of NUN 07839 LTL. The “seed grown on NUN 07839 LTL” contains tissues and DNA of the maternal parent, NUN 07839 LTL. The “seed grown on NUN 07839 LTL” contains an F1 embryo. When said seed is planted, it grows into a first generation progeny plant of NUN 07839 LTL.

“USDA descriptors” are the plant variety descriptors described for lettuce in the “Objective description of Variety—Lettuce (Lactuca sativa L.)”, as published by U.S. Department of Agriculture, Agricultural Marketing Service, Science and Technology, Plant Variety Protection Office, Beltsville, Md. 20705 and which can be downloaded from the world wide web at ams.usda.gov/ under sites/default/files/media/01-Lettuce %20ST-470-01%202015.pdf. “Non-USDA descriptors” are other descriptors suitable for describing lettuce

“UPOV descriptors” are the plant variety descriptors described for lettuce in the “Guidelines for the Conduct of Tests for Distinctness, Uniformity and Stability,” TG/013/10 (Geneva 2006, last updated Mar. 20, 2013), as published by UPOV (International Union for the Protection of New Varieties and Plants) and which can be downloaded from the world wide web at upov.int/ under edocs/tgdocs/en/tg013.pdf and is herein incorporated by reference in its entirety. Likewise, “UPOV methods” to determine specific parameters for the characterization of lettuce are described at upov.int.

“RHS” refers to the Royal Horticultural Society of England which publishes an official botanical color chart quantitatively identifying colors according to a defined numbering system, The chart may be purchased from Royal Horticulture Society Enterprise Ltd RHS Garden; Wisley, Woking; Surrey GU236QB, UK, e.g., the RHS colour chart: 2007 (The Royal Horticultural Society, charity No: 222879, PO Box 313 London SW1P2PE; sold by, e.g., TORSO-VERLAG, Obere Grüben 8 ⋅ D-97877 Wertheim, Article-No.: Art62-00008 EAN-Nr.: 4250193402112).

As used herein and except as otherwise indicated, the term “plant” includes the whole plant or any part thereof, preferably having the same genetic makeup as the plant from which it is obtained, such as a plant organ (e.g. harvested or non-harvested fruits), a plant cell, a plant protoplast, a plant cell tissue culture or a tissue culture from which a whole plant can be regenerated, a plant cell that is intact in a plant, a clone, a micropropagation, plant callus, a plant cell clump, a plant transplant, a vegetative propagation, a seedling, or parts of a plant (e.g. harvested tissues or organs), such as a fruit, a harvested fruit, a part of a fruit, a leaf, a part of a leaf, pollen, an ovule, an embryo, a petiole, a shoot or a part thereof, a stem or a part thereof, a root or a part thereof, a root tip, a cutting, a seed, a part of a seed, seed coat or another maternal tissue which is part of a seed grown on a variety of the invention, hypocotyl, cotyledon, a scion, a graft, a stock, a rootstock, a pistil, an anther, and a flower or parts of any of these and the like. Also any developmental stage is included, such as seedlings, cuttings prior or after rooting, mature plants or leaves. Alternatively, a plant part may also include a plant seed which comprises one or two sets of chromosomes derived from the parent plant; e.g. from NUN 09102 LTL. An F1 progeny produced from self-pollination of the inbred variety NUN 09102 LTL will thus comprise two sets of chromosomes derived from NUN 09102 LTL, while an F1 progeny derived from cross-fertilization of

NUN 09102 LTL will comprise only one set of chromosomes from NUN 09102 LTL and the other set of chromosomes from the other parent.

“Cotyledon” refers to one of the first leaves of the embryo of a seed plant; typically one or more in monocotyledons, two in dicotyledons and two or more in gymnosperms.

“Head” as used herein refers to lettuce heads, i.e., the plant without the root system, for example substantially all harvested leaves. Encompassed are immature leaves (e.g. “baby leaf”) and mature leaves.

The “base” of a plant is the part of a lettuce plant where the leaves are attached to the root system of the plant.

“Core length” of the internal lettuce stem is measured from the base of the cut and trimmed head to the tip of the stem.

“Head weight” refers to the mean weight of saleable lettuce head, cut and trimmed to market specifications.

“Head diameter” refers to the mean diameter of the cut and trimmed head, sliced vertically, and measured at the widest point perpendicular to the stem.

“Head height” refers to the mean height of the cut and trimmed head, sliced vertically, and measured from the base of the cut stem to the leaf tip.

“Core Length to Head Diameter Ratio (CLHD Ratio)” refers to the mean core length/head diameter ratio. It is calculated by dividing the mean core length with the mean head diameter. This is an indication of the head shape and of the ability of a lettuce plant to reduce the amount of surface which is on or close to the ground.

“Harvested plant material” refers herein to plant parts (e.g., leaves or heads detached from the whole plant) which have been collected for further storage and/or further use.

“Harvested seeds” refers to seeds harvested from a line or variety, e.g., produced after self-fertilization or cross-fertilization and collected.

“REFERENCE VARIETY” refers to variety ______, a commercial variety from company ______, which has been planted in a trial together with NUN 07839 LTL. USDA descriptors of NUN 07839 LTL were compared to the USDA descriptors of ______.

“Yield” means the total weight of all lettuce heads or leaves harvested per hectare of a particular line or variety. It is understood that “yield” expressed as weight of all lettuce heads or leaves harvested per hectare can be obtained by multiplying the number of plants per hectare times the “yield per plant”.

“Marketable yield” means the total weight of all marketable lettuce heads or leaves harvested per hectare of a particular line or variety, i.e. lettuce heads or leaves suitable for being sold for fresh consumption, having good color, glossiness size and texture and no or very low levels of deficiencies.

A plant having “all the physiological and morphological characteristics” of a referred-to-plant means a plant having or showing the physiological and morphological characteristics of the referred-to-plant when grown under the same environmental conditions, preferably in the same experiment next to each other; the referred-to-plant can be a plant from which it was derived, e.g. the progenitor plant, the parent, the recurrent parent, the plant used for tissue- or cell culture, etc. A physiological or morphological characteristic can be a numerical characteristic or a non-numerical characteristic. In one aspect, a plant has “all but one, two or three of the physiological and morphological characteristics” of a referred-to-plant, or “all the physiological and morphological characteristics” of Table 1 and/or 2 or “all or all but one, two or three of the physiological and morphological characteristics” of Table 1 and/or 2.

NUN 09102 LTL has the following distinguishing characteristics when compared to the Reference Variety 1) ______; 2) ______; 3) ______; 4) ______; 5) ______; 6) ______; 7) ______; 8) ______; 9) ______; and 10) ______. This can be seen in among others Table 1, where the USDA characteristics of NUN 09102 LTL are compared to Reference Variety, when grown under the same environmental conditions.

In certain embodiments the plant of the invention has all the physiological and morphological characteristics, except for certain characteristics mentioned, e.g. the characteristic(s) derived from a converted or introduced gene or trait and/or except for the characteristics which differ, for example a Single Locus Conversion or a mutation.

In one embodiment, the invention relates to a Single Locus Converted plant or a mutated plant of NUN 07839 LTL.

Similarity and differences between two different plant lines or varieties can be determined by comparing the number of morphological and/or physiological characteristics (e.g. the characteristics as listed in Table 1 and/or 2) that are the same (i.e. statistically not significantly different) or that are different (i.e. statistically significantly different) between the two plant lines or varieties when grown under the same environmental conditions. A numerical characteristic is considered to be “the same” when the value for a numeric characteristic is not significantly different at the 1% (p<0.01) or 5% (p<0.05) significance level, using one way Analysis of variance (ANOVA), a standard method known to the skilled person. Non-numerical or “degree” or “type” characteristic are considered “the same” when the values have the same “degree” or “type” when scored using USDA and/or UPOV descriptors, if the plants are grown under the same environmental conditions.

“Distinguishing characteristics” or “distinguishing morphological and/or physiological characteristics” refers herein to the characteristics which distinguish (i.e. are different) between the new variety and other lettuce varieties, such as the REFERENCE VARIETY, when grown under the same environmental conditions. The distinguishing characteristics between NUN 09102 LTL and REFERENCE VARIETY are described elsewhere herein and can be seen in Table 1 and/or Table 2. When comparing NUN 09102 LTL with different varieties, the distinguishing characteristics will be different. In one aspect, the distinguishing characteristics may therefore include at least one, two, three or more (or all) of the characteristics listed in Table 1 and/or 2. All numerical distinguishing characteristics are statistically significantly different at p<0.05 between NUN 09102 LTL and the other variety, e.g. REFERENCE VARIETY.

Thus, a lettuce plant “comprising the distinguishing characteristics of NUN 09102 LTL (such as a progeny plant) refers herein to a plant which does not differ significantly from said variety in characteristics 1) to 10) above. Therefore in one aspect a plant (such as a progeny plant of NUN 09102 LTL) is provided which does not differ significantly from NUN 09102 LTL in the distinguishing characteristics 1) to 10) above.

The physiological and/or morphological characteristics mentioned above are commonly evaluated at significance levels of 1%, 5% or 10% if they are numerical, or for being an identical type or degree if not numerical, when measured under the same environmental conditions. For example, a progeny plant or a Single Locus Converted plant or a mutated plant of NUN 07839 LTL may have one or more (or all) of the essential physiological and/or morphological characteristics of said variety listed in Table 1 and/or 2, as determined at the 5% significance level (i.e. p<0.05) when grown under the same environmental conditions.

As used herein, the term “variety”, “cultivated lettuce” or “cultivar” means a plant grouping within a single botanical taxon of the lowest known rank, which grouping, irrespective of whether the conditions for the grant of a breeder's right are fully met, can be defined by the expression of the characteristics resulting from a given genotype or combination of genotypes, distinguished from any other plant grouping by the expression of at least one of the said characteristics and considered as a unit with regard to its suitability for being propagated unchanged.

“Plant line” is for example an inbred variety or a breeding line which can be used to develop one or more varieties. Both are typically highly homozygous. Progeny obtained by selfing such a plant line has the same phenotype as its parents.

“Inbred variety” refers to an inbred (nearly homozygous) line or seeds thereof. For example, the (nearly homozygous) plant is self-pollinated or the (nearly homozygous) female parent is pollinated with pollen of the same plant line to produce inbred seeds on the female parent.

“Tissue Culture” refers to a composition comprising isolated cells of the same or a different type or a collection of such cells organized into parts of a plant. Tissue culture of various tissues of lettuce and regeneration of plants therefrom is well known and widely published (see, e.g., Teng et al., HortScience. 1992, 27(9): 1030-1032 Teng et al., HortScience. 1993, 28(6): 669-1671, Zhang et al., Journal of Genetics and Breeding. 1992, 46(3): 287-290). Similarly, the skilled person is well-aware how to prepare a “cell culture”.

“Vegetative propagation”, “vegetative reproduction” or “clonal propagation” are used interchangeably herein and mean the method of taking part of a plant and allowing that plant part to form at least roots where plant part is, e.g., defined as or obtained from (e.g. by cutting of) a fruit, a harvested fruit, a part of a fruit, a leaf, a part of a leaf, pollen, an ovule, a cell, a petiole, a shoot or a part thereof, a stem or a part thereof, a root or a part thereof, a root tip, a cutting, a seed, hypocotyl, cotyledon, a scion, a stock, a rootstock, a pistil, an anther, and a flower or a part thereof, etc. When a whole plant is regenerated by vegetative propagation, it is also referred to as a vegetative propagation.

“Regeneration” refers to the development of a plant from cell culture or tissue culture or vegetative propagation.

“Crossing” refers to the mating of two parent plants. The term encompasses “cross-pollination” and “selfing”. “Cross-pollination” refers to the fertilization by the union of two gametes from different plants

“Selfing” refers to self-pollination of a plant, i.e., the transfer of pollen from the anther to the stigma of the same plant. Lettuce is an obligate self-pollination species, which means that pollen is shed before stigma emergence, assuring 100% self-fertilization. Therefore, in order to optimize crossing, a method of misting may be used to wash the pollen off prior to fertilization to assure crossing or hybridization.

As used herein, the terms “resistance” and “tolerance” are used interchangeably to describe plants that show no symptoms or significantly reduced symptoms to a specified biotic pest, pathogen, abiotic influence or environmental condition compared to a susceptible plant. These terms are optionally also used to describe plants showing some symptoms but that are still able to produce marketable product with an acceptable yield.

“Locus” (plural loci) refers to the specific location of a gene or DNA sequence on a chromosome, where, for example, a gene or genetic marker is found. A locus may confer a specific trait.

“Genotype” refers to the genetic composition of a cell or organism.

“Allele” refers to one or more alternative forms of a gene locus. All of these loci relate to one trait. Sometimes, different alleles can result in different observable phenotypic traits, such as different pigmentation. However, many variations at the genetic level result in little or no observable variation. If a multicellular organism has two sets of chromosomes, i.e. diploid, these chromosomes are referred to as homologous chromosomes. Diploid organisms have one copy of each gene (and therefore one allele) on each chromosome. If both alleles are the same, they are homozygotes. If the alleles are different, they are heterozygotes.

The term “traditional breeding techniques” encompasses herein crossing, selfing, selection, doubled haploid production, embryo rescue, protoplast fusion, marker assisted selection, mutation breeding etc. as known to the breeder (i.e. methods other than genetic modification/transformation/transgenic methods), by which, for example, a genetically heritable trait can be transferred from one lettuce line or variety to another.

“Progeny” as used herein refers to a plant obtained from a plant designated NUN 07839 LTL. A progeny may be obtained by regeneration of cell culture or tissue culture or parts of a plant of said variety or selfing of a plant of said variety (such a progeny is an identical clone of the parent) or by producing seeds of a plant of said variety (such a progeny is not genetically identical to the parent, unless both parents are of the same highly homozygous line). In further embodiments, progeny may also encompass plants obtained from crossing of at least one plant of said variety with another lettuce plant of the same or another variety or (breeding) line, or wild lettuce plants, backcrossing, inserting of a locus into a plant or mutation. A progeny can be a first generation progeny, i.e. the progeny is directly derived from, obtained from, obtainable from or derivable from the parent plant by, e.g., traditional breeding methods (selfing and/or cross-pollinating) or regeneration. However, the term “progeny” generally encompasses further generations such as second, third, fourth, fifth, sixth, seventh or more generations, i.e., generations of plants which are derived from, obtained from, obtainable from or derivable from the former generation by, e.g., traditional breeding methods, regeneration or genetic transformation techniques. For example, a second generation progeny can be produced from a first generation progeny by any of the methods mentioned above.

The terms “gene converted” or “conversion plant” in this context refer to lettuce plants which are developed by backcrossing wherein essentially all of the desired morphological and physiological characteristics of parent are recovered in addition to the one or more genes transferred into the parent via the backcrossing technique or via genetic engineering. Likewise a “Single Locus Converted (Conversion) Plant” refers to plants which are developed by plant breeding techniques comprising or consisting of backcrossing, wherein essentially all of the desired morphological and physiological characteristics of a lettuce variety are recovered in addition to the characteristics of the single locus having been transferred into the variety via the backcrossing technique and/or by genetic transformation and/or by mutation.

“Transgene” or “chimeric gene” refers to a genetic locus comprising a DNA sequence which has been introduced into the genome of a lettuce plant by transformation. A plant comprising a transgene stably integrated into its genome is referred to as “transgenic plant”.

“Marker” refers to a readily detectable phenotype, preferably inherited in codominant fashion (both alleles at a locus in a diploid heterozygote are readily detectable), with no environmental variance component, i.e., a heritability of 1.

“Substantially equivalent” refers to a characteristic that, when compared, does not show a statistically significant difference (e.g., p=0.05) from the mean.

It is understood that “significant” differences refer to statistically significant differences, when comparing the characteristic between two plant lines or varieties when grown under the same conditions. Preferably at least about 10, 15, 20 or more plants per line or variety are grown under the same conditions (i.e. side by side) and characteristics are measured on at least about 10, 15, 20 or more randomly selected plant or plant parts to obtain averages. Thus, physiological and morphological characteristics or traits are commonly evaluated at a significance level of 1%, 5% or 10%, when measured in plants grown under the same environmental conditions. Alternatively, “significance” or “statistical significance” of differences can be expressed as a p-value. A p-value represents the probability of obtaining a result equal to or more extreme than the result actually observed. ANOVA is a suitable method for determining the value of p (Clewer, A. G., and D. H. Scarisbrick. 2001 contains a comprehensive explanation of the whole comparison process). Thus, physiological and morphological characteristics or traits are commonly evaluated at a significance level of p<0.1, or preferably p<0.05or even more preferably p<0.01 when measured in plants grown under the same environmental condition.

“Average” refers herein to the arithmetic mean.

The term “mean” refers to the arithmetic mean of several measurements. The skilled person understands that the appearance of a plant depends to some extent on the growing conditions of said plant. Thus, the skilled person will know typical growing conditions for a lettuce variety. The mean, if not indicated otherwise within this application, refers to the arithmetic mean of measurements on at least 10 different, randomly selected plants of a variety or line.

### DETAILED DESCRIPTION

The present invention relates to a lettuce (Lactuca sativa) variety, designated NUN 07839 LTL, which—when compared to its REFERENCE VARIETY ______—has: 1) ______; 2) ______; 3) ______; 4) ______; 5) ______; 6) ______; 7) ______; 8) ______; 9) ______; and 10) ______, when grown under the same conditions. Also encompassed by the present invention are progeny plants having all or all but 1, 2, or 3 of the morphological and/physiological characteristics of NUN 07839 LTL and methods of producing plants in accordance with the present invention.

Thus, in one aspect, the invention provides a seed of the variety designated NUN 07839 LTL wherein a representative sample of seeds of said variety was deposited under the Budapest Treaty, with Accession number NCIMB 42969.

In another aspect, the invention provides for a plant of variety NUN 07839 LTL, or a head or a leaf or another plant part thereof, including a tissue or cell culture, a representative sample of seed from said variety has been deposited under the Budapest Treaty, with Accession number NCIMB 42969.

A seed of inbred variety NUN 07839 LTL is obtainable by self-pollinating NUN 07839 LTL and harvesting the seeds produced. The resultant seeds of said variety can be grown to produce plants of said variety. In one embodiment a seed or a plurality of seeds of said variety are packaged into containers of any size or type (e.g., bags, cartons, cans, etc.). The seed may be disinfected, primed and/or treated with various compounds, such as seed coatings or crop protection compounds.

It is understood that a tissue or cell culture of NUN 07839 LTL can be obtained from any plant part of said variety. In one embodiment the invention provides a lettuce plant regenerated from the tissue or cell culture of NUN 07839 LTL, wherein the regenerated plant is not significantly different from NUN 07839 LTL in all, or all but one, two or three, of the physiological and morphological characteristics (determined at the 5% significance level or evaluated at p<0.05 using ANOVA when grown under the same conditions. In another embodiment, the invention provides a lettuce plant regenerated from the tissue or cell culture of NUN 07839 LTL, wherein the plant has all or all but one, two or three of the physiological and morphological characteristics of said variety. In these cases, similarity or difference of a characteristic is determined by measuring the characteristics of a representative number of plants grown under the same environmental conditions, determining whether type/degree characteristics are the same or different and determining whether numerical characteristics are significantly different (determined at the 5% significance level or evaluated at p<0.05 using ANOVA.

In other aspects, the invention provides for a head or a part thereof or a leaf or a part thereof of variety NUN 07839 LTL, or a plant part, such as pollen, flowers, shoots or cuttings of said variety or parts thereof

A plant of NUN 07839 LTL can be produced by seeding directly in the soil (e.g., field) or by germinating the seeds in controlled environment conditions (e.g., greenhouses) and then transplanting the seedlings into the field (see, e.g., Gonai et al., J. of Exp. Bot., 55(394): 111, 2004; Louise Jackson et al, Publication 7215 ISBN 978-1-60107-007-4 and Publication 7216 ISBN 978-1-60107-008-1 and the world wide web at “anrcatalog.ucdavis.edu” search: lettuce for cultivation, harvesting, handling and postharvest methods commonly used). Lettuce may also be grown in tunnels. Moreover, said variety can be grown in hydroponic cultures as described in, e.g., US 2008/0222949, and the skilled person is familiar with various types of hydroponic cultures. Alternatively, seed of said variety may be grown on peat block for use as root ball lettuce. Furthermore, said variety may be combined with 1, 2 or 3 different lettuce varieties to be grown as “composite lettuce” (see, e.g., EP 1 197 137 A1).

In one embodiment a plant of NUN 07839 LTL comprises at least 3, 4, 5 or more, e.g. 6, 7, 8, 9 or all of the following morphological and/or physiological characteristics when grown under the same environmental conditions (i.e. average values of distinguishing characteristics, as indicated on the USDA Objective description of variety—Lettuce (unless indicated otherwise)): 1) ______; 2) ______; 3) ______; 4) ______; 5) ______; 6) ______; 7) ______; 8) ______; 9) ______; and 10) ______.

NUN 07839 LTL may further exhibit at least one further trait selected from the group consisting of a) ______, b) ______, c) ______, d) ______.

In another embodiment NUN 07839 LTL has resistance to ______ that is ______ on a scale of ______; and it has resistance to ______ that is ______ on a scale of ______.

In still another aspect the invention provides a method of producing a lettuce plant, comprising crossing a plant of lettuce variety NUN 07839 LTL with a second lettuce plant one or more times, and selecting progeny from said crossing. In one embodiment of the invention, the first step in “crossing” comprises planting seeds of a first and a second parent lettuce plant, often in proximity so that pollination will occur for example, mediated by insect vectors. Alternatively, pollen can be transferred manually. Where the plant is self-pollinated, pollination may occur without the need for direct human intervention other than plant cultivation.

In yet another aspect the invention provides a method of producing a plant, comprising selfing a plant of variety NUN 07839 LTL one or more times, and selecting a progeny plant from said selfing. In one aspect the progeny plant retains all the distinguishing characteristics of NUN 07839 LTL described above. In a different embodiment the progeny plant comprises all (or all but one, two or three) of the physiological and morphological characteristic of NUN 07839 LTL of Table 1, and/or Table 2. In a further embodiment the progeny plant comprises all physiological and morphological characteristic of NUN 07839 LTL when grown under the same environmental conditions.

In other aspects, the invention provides for a progeny plant of variety NUN 07839 LTL such as a progeny plant obtained by further breeding that variety. Further breeding with the variety of the invention includes selfing that variety one or more times and/or cross-pollinating that variety with another lettuce plant or variety one or more times. In particular, the invention provides for a progeny plant that retains all the essential morphological and physiological characteristics of that variety or, in another embodiment, progeny that retain all or all but one, two or three of the morphological and physiological characteristics of NUN 07839 LTL, optionally all or all but one, two or three of the morphological and physiological characteristics listed in Table 1 and/or 2, when grown under the same environmental conditions, determined at the 5% significance level for numerical characteristics. In another aspect, the invention provides for a vegetative reproduction of the variety and a plant having all or all but 1, 2, or 3 of the physiological and morphological characteristics of NUN 07839 LTL.

The morphological and/or physiological differences between two different individual plants of the invention (e.g. between NUN 07839 LTL and a progeny of NUN 07839 LTL) or progeny of said variety, or a plant having all, or all but 1, 2, or 3 of the physiological and morphological characteristics of NUN 07839 LTL (or all, or all but 1, 2, or 3 of the characteristics as listed in Table 1 and/or 2) and another known variety can easily be established by growing said variety NUN 07839 LTL next to the other variety (in the same field, under the same environmental conditions), preferably in several locations which are suitable for said lettuce cultivation, and measuring morphological and/or physiological characteristics of a number of plants (e.g., to calculate an average value and to determine the variation range/uniformity within the variety). For example, trials can be carried out in Acampo Calif., USA (N 38 degrees 07′261″/W 121 degrees 18′ 807″, USA, whereby various characteristics, for example maturity, leaf shape, size and texture, leaf color and glossiness, bolt shape, surface and length, flower size and color, head weight, disease resistance, insect resistance and resistance to physiological stress can be measured and directly compared for species of Lactuca sativa.

The morphological and physiological characteristics (and the distinguishing characteristics) of NUN 07839 LTL are provided in the Examples, in Table 1 and/or 2. Encompassed herein is also a plant obtainable from NUN 07839 LTL (e.g. by selfings and/or crossing and/or backcrossing with said variety and/or progeny of said variety) comprising all or all but one, two or three of the physiological and morphological characteristics of NUN 07839 LTL listed in Table 1 and/or 2 as determined at the 5% significance level for numerical characteristics or identical for non-numerical characteristics when grown under the same environmental conditions and/or comprising one or more (or all; or all except one, two or three) when grown under the same environmental conditions.

Also at-harvest and/or post-harvest characteristics of heads or leaves can be compared, such as cold storage holding quality, post-harvest leaf crispness and leaf browning or pinking after cutting can be measured using known methods.

The morphological and/or physiological characteristics may vary somewhat with variation in the environment (such as temperature, light intensity, day length, humidity, soil, fertilizer use), which is why a comparison under the same environmental conditions is preferred. Colors can best be measured against The Munsell Book of Color (Munsell Color Macbeth Division of Kollmorgan Instruments Corporation) or using the Royal Horticultural Society Chart (World wide web at rhs.org.uk/Plants/RHS-Publications/RHS-colour-charts).

In a preferred embodiment, the invention provides for a lettuce head or leaf of variety NUN 07839 LTL, or a part of the head or a leaf or a part of a leaf. In another embodiment, the invention provides for a container comprising or consisting of a plurality of harvested lettuce heads or leaves of said variety, or of progeny thereof.

In yet a further embodiment, the invention provides for a method of producing a new lettuce plant. The method comprises crossing a plant of the invention i.e. NUN 07839 LTL, or a plant comprising all but 1, 2, or 3 of the morphological and physiological characteristics of said variety (as listed in Table 1 and/or 2), or a progeny plant thereof, either as male or as female parent, with a second lettuce plant (or a wild relative of lettuce) one or more times, and/or selfing a lettuce plant according to the invention i.e. NUN 07839 LTL, or a progeny plant thereof, one or more times, and selecting progeny from said crossing and/or selfing. The second lettuce plant may for example be a line or variety of the species Lactuca sativa, or other Lactuca species or even other Asteraceae species.

Progeny are either the generation (of seeds) produced from the first cross of a lettuce variety of the invention with another plant (F1) or with itself (S1), or any further generation produced by crossing and/or selfing (F2, F3, etc.) and/or backcrossing (BC1, BC2, etc.) one or more selected plants of the F1 and/or S1 and/or BC1 generation (or plants of any further generation, e.g. the F2) with another lettuce plant (and/or with a wild relative of lettuce). Progeny can also refer to a vegetative propagation or a regenerated plant of the invention. Progeny may have all the physiological and morphological characteristics of lettuce variety NUN 07839 LTL when grown under the same environmental conditions and/or progeny may have (be selected for having) one or more of the distinguishing characteristics of lettuce of the invention. Using common breeding methods such as backcrossing or recurrent selection, one or more specific characteristics may be introduced into said variety, to provide or a plant comprising all but 1, 2, or 3 or more of the morphological and physiological characteristics of NUN 07839 LTL (as listed in Table 1 and/or 2).

The invention provides for methods of producing plants which retain all the morphological and physiological characteristics of a plant of the invention i.e. NUN 07839 LTL The invention provides also for methods of producing a plant comprising all but 1, 2, or 3 or more of the morphological and physiological characteristics of NUN 07839 LTL as listed in Table 1 and/or 2), but which are still genetically closely related to said variety. The relatedness can, for example be determined by fingerprinting techniques (e.g., making use of isozyme markers and/or molecular markers such as Single-nucleotide polymorphism (SNP) markers, amplified fragment length polymorphism (AFLP) markers, microsatellites, minisatellites, Random Amplified Polymorphic DNA (RAPD) markers, restriction fragment length polymorphism (RFLP) markers and others). A plant is “closely related” to NUN 07839 LTL if its DNA fingerprint is at least 80%, 90%, 95% or 98% identical to the fingerprint of NUN 07839 LTL. In a preferred embodiment AFLP markers are used for DNA fingerprinting (Vos et al. 1995, Nucleic Acid Research 23: 4407-4414). A closely related plant may have a Jaccard's Similarity index of at least about 0.8, preferably at least about 0.9, 0.95, 0.98 or more.

The invention also provides a plant and a variety obtained or selected by applying these methods on NUN 07839 LTL. Such a plant may be produced by crossing and/or selfing, or alternatively, a plant may simply be identified and selected amongst plants of said variety, or progeny of said variety, e.g. by identifying a variant within NUN 07839 LTL or progeny of said variety (e.g. produced by selfing) which variant differs from NUN 07839 LTL in one, two or three of the morphological and/or physiological characteristics (e.g. in one, two or three distinguishing characteristics), e.g. those listed in Table 1 and/or 2 or others. In one embodiment the invention provides a lettuce plant having a Jaccard's Similarity index with NUN 07839 LTL of at least 0.8, e.g. at least 0.85, 0.9, 0.95, 0.98 or even at least 0.99.

The present invention also provides a lettuce seed and a plant produced by a process that comprises crossing a first parent lettuce plant with a second parent lettuce plant, wherein at least one of the first or second parent lettuce plants is a plant provided herein, such as from variety NUN 07839 LTL. In another embodiment of the invention, lettuce seed and plants produced by the process are first filial generation (F1) lettuce seed and plants produced by crossing a plant in accordance with the invention with another, distinct plant.

The present invention further contemplates plant parts of such an F1 lettuce plant, and methods of use thereof. Therefore, certain exemplary embodiments of the invention provide an F1 lettuce plant and seed thereof.

WO2013182646 which is incorporated by reference, relates to a non-destructive method for analyzing maternal DNA of a seed. In this method the DNA is dislodged from the seed coat surface and can be used to collect information on the genome of the maternal parent of the seed. This method for analyzing maternal DNA of a seed, comprises the steps of contacting a seed with a fluid to dislodge DNA from the seed coat surface, and analyzing the DNA thus dislodged from the seed coat surface using methods known in the art. The skilled person is thus able to determine whether a seed has grown on a plant of a plant of the invention i.e. NUN 07839 LTL is a progeny of said variety, because the seed coat of the seed is a maternal tissue genetically identical to NUN 07839 LTL. In one embodiment, the present invention relates to a seed coat comprising maternal tissue of NUN 07839 LTL. In another embodiment the invention relates to a lettuce seed comprising a maternal tissue of NUN 07839 LTL.

By crossing and/or selfing also (one or more) single traits may be introduced into the variety of the invention i.e. NUN 07839 LTL (e.g., using backcrossing breeding schemes), while retaining the remaining morphological and physiological characteristics of said variety and/or while retaining one or more distinguishing characteristics. A single trait converted plant may thereby be produced. For example, disease resistance genes may be introduced, genes responsible for one or more quality traits, yield, etc. Both single genes (dominant or recessive) and one or more QTLs (quantitative trait loci) may be transferred into NUN 07839 LTL by breeding with said variety.

Any trait can be introduced. In a preferred embodiment, pest or disease resistance genes may be introduced into a plant according to the invention, i.e. NUN 07839 LTL, progeny of said variety or into a plant comprising all but 1, 2, or 3 or more of the morphological and physiological characteristics of NUN 07839 LTL (e.g. as listed in Table 1 and/or 2). Resistance to one or more of the following diseases or pests is preferably introduced into plants of the invention: Downy mildew, Powdery mildew, Sclerotinia rot, Sclerotinia drop, Botrytis (Grey Mold), Verticillium Wilt, Pseudomonas spp. (Bacterial Soft Rot), Bacterial Leaf Spot, Anthracnose, Bottom rot, Corky root rot, Lettuce mosaic virus, Turnip mosaic virus, Tomato bushy stunt virus (Dieback), Big vein, Cabbage Loopers, Root Aphid, Green Peach Aphid, Lettuce aphid, Pea leafminer, Beet western yellows and aster yellows. Other resistance genes, against pathogenic viruses (e.g. Lettuce infectious yellows virus (LIYV), lettuce mosaic virus (LMV), Cucumber mosaic virus (CMV), Beet western yellows virus (BWYV), Alfalfa mosaic virus (AMV)), fungi, bacteria or lettuce pests may also be introduced. In one embodiment resistance against Nasonovia ribisnigri biotype Nr:0 and/or Nr:1 is introduced a plant of the invention. Other resistance genes, against pathogenic viruses, fungi, bacteria, nematodes, insects or other pests may also be introduced. Also, any resistances to physiological stresses may be introduced into a plant according to the invention, or progeny thereof or into a plant comprising all but 1, 2, or 3 or more of the morphological and physiological characteristics of said plant (e.g. as listed in Table 1 and/or 2. Resistance against one or more of the following is preferably introduced into plants of the invention: Tipburn, Heat, Drought, Cold, Salt and/or Brown rob (Rib discoloration/rib blight).

Thus, invention also provides a method for developing a plant in a breeding program, using a lettuce plant of the invention, or its parts as a source of plant breeding material. Suitable plant breeding techniques are recurrent selection, backcrossing, pedigree breeding, mass selection, mutation breeding and/or genetic marker enhanced selection. For example, in one aspect, the method comprises crossing NUN 07839 LTL or progeny of said variety, or a plant comprising all but 1, 2, or 3 or more of the morphological and physiological characteristics of NUN 07839 LTL (e.g. as listed in Table 1 and/or 2) with a different lettuce plant, and wherein one or more offspring of the crossing are subject to one or more plant breeding techniques selected from the group consisting of recurrent selection, backcrossing, pedigree breeding, mass selection, mutation breeding and genetic marker enhanced selection (see e.g. Brotman et al., Theor Appl Genet (2002) 104:1055-1063). Pedigree selection, also known as the “Vilmorin system of selection,” is described in, e.g., Allard, 1960, John Wiley & Sons, Inc.: Principles of plant breeding: 119-128, Library of Congress Catalog Card Number: 60-14240. For breeding methods in general see Principles of Plant Genetics and Breeding, 2007, George Acquaah, Blackwell Publishing, ISBN-13: 978-1-4051-3646-4.

Thus, in one aspect a method for developing a lettuce plant in a lettuce breeding program is provided, using a lettuce plant of the invention, or its parts, as a source of plant breeding material. Suitable plant breeding techniques are recurrent selection, backcrossing, pedigree breeding, line selection, mass selection, mutation breeding and/or genetic marker enhanced selection. For example, in one aspect, the method comprises crossing a lettuce plant designated NUN 07839 LTL, or progeny thereof, or a plant comprising all but 1, 2, or 3 or more of the morphological and physiological characteristics of said plant variety (e.g. as listed in Table 1 and/or 2), with a different lettuce plant selected from the group consisting of a plant of the same variety, a lettuce plant of a different variety, a (breeding) line, or a wild relative of lettuce (e.g., L. virosa or L. serriola), and wherein one or more offspring of the crossing are subject to one or more plant breeding techniques selected from the group consisting of recurrent selection, backcrossing, pedigree breeding, mass selection, mutation breeding and genetic marker enhanced selection (see e.g. Dziechciarková et al, PLANT SOIL ENVIRON., 50, 2004 (2): 47-58). For breeding methods in general see Principles of Plant Genetics and Breeding, 2007, George Acquaah, Blackwell Publishing, ISBN-13: 978-1-4051-3646-4 or Principles of plant breeding, 1960, Allard, John Wiley & Sons, Inc,: Library of Congress Catalog Card Number: 60-14240).

Alternatively, a single trait converted plant or single locus converted plant of NUN 07839 LTL may be produced by the following steps

a. obtaining a cell or tissue culture of cells of NUN 07839 LTL;

b. genetically transforming or mutating said cells;

c. growing the cells into a plant; and

d. optionally selecting a plant that contains the desired single locus conversion

The skilled person is familiar with various techniques for genetically transforming a single locus in a plant cell, or mutating said cells.

In another aspect the invention provides a method of introducing a single locus conversion or introducing a desired trait into a plant designated NUN 07839 LTL, comprising:
(a) crossing the plant designated NUN 07839 LTL, representative seed of which having been deposited under Accession Number NCIMB 42969, with a second plant comprising a desired single locus to produce F1 progeny plants and obtaining progeny of said crossing; wherein the single locus comprised by the second plant is the locus to be introduced in the first plant;
(b) optionally selfing said F1 progeny plant to produce an F2 progeny plant having said single locus;

Said method may be followed by a step of
(c) crossing the progeny plant with the plant designated NUN 07839 LTL, representative seed of which having been deposited under Accession Number NCIMB 42969, and obtaining progeny of said cross;
(d) optionally repeating step (c) one or more times in succession to produce selected second or higher backcross progeny plants that comprise the single locus and otherwise comprise essentially all physiological and morphological characteristics when grown under the same environmental conditions of a plant designated NUN 07839 LTL.

The invention is also directed to a lettuce plant obtained from step a), b), c) or d) of the above method.

Any trait can be introduced. In one embodiment the trait to be used in the above method for single locus conversion is disease resistance and the resistance is conferred to any race of Nasonovia ribisnigri, any race of Downy mildew, Powdery mildew, Sclerotinia rot, Sclerotinia drop, Botrytis (Grey Mold), Verticillium Wilt, Pseudomonas spp.(Bacterial Soft Rot), Bacterial Leaf Spot, Anthracnose, Bottom rot, Corky root rot, Lettuce mosaic virus, Turnip mosaic virus, Tomato bushy stunt virus (Dieback), Big vein, Cabbage Loopers, Root Aphid, Green Peach Aphid, Lettuce aphid, Pea leafminer, Beet western yellows and aster yellows, Sclerotinia minor (leaf drop), Sclerotinia sclerotiorum (leaf drop), Rhizoctonia solani (bottom drop), Erysiphe cichoracearum (powdery mildew), Fusarium oxysporum f. sp. Lactucae (Fusarium wilt), lettuce infectious yellows virus (LIYV), lettuce mosaic virus (LMV), Cucumber mosaic virus (CMV), Beet western yellows virus (BWYV), and Alfalfa mosaic virus (AMV).

The invention also provides a lettuce plant comprising at least a first set of the chromosomes of lettuce variety NUN 07839 LTL, a sample of seed of said variety having been deposited under Accession Number NCIMB 42969; optionally further comprising a single locus conversion or a mutation, wherein said plant has essentially all of the morphological and physiological characteristics of the plant comprising at least a first set of the chromosomes of said variety. In another embodiment, this single locus conversion confers a trait selected from the group consisting of yield, storage properties, color, male sterility, herbicide tolerance, insect resistance, pest resistance, disease resistance, environmental stress tolerance, modified carbohydrate metabolism and modified protein metabolism.

In one embodiment, a plant according to the invention, i.e. NUN 07839 LTL or NUN 05379 LTL may also be mutated (by e.g. irradiation, chemical mutagenesis, heat treatment, etc.) and mutated seeds or plants may be selected in order to change one or more characteristics of said variety. Methods such as TILLING may be applied to lettuce populations in order to identify mutants. Similarly, NUN 07839 LTL may be transformed and regenerated, whereby one or more chimeric genes are introduced into the variety. Transformation can be carried out using standard methods, such as Agrobacterium tumefaciens mediated transformation or biolistics, followed by selection of the transformed cells and regeneration into plants. A desired trait (e.g. genes conferring pest or disease resistance, herbicide, fungicide or insecticide tolerance, etc.) can be introduced into NUN 07839 LTL, or progeny of said variety, by transforming said variety or progeny of said variety with a transgene that confers the desired trait, wherein the transformed plant retains all or all but one, two or three of the phenotypic and/or morphological and/or physiological characteristics of NUN 07839 LTL or the progeny of said variety and contains the desired trait.

The invention also provides a plant or a cell of a plant comprising a desired trait produced by mutating a plant of variety NUN 07839 LTL or a cell thereof and selecting a plant the desired trait, wherein the mutated plant retains all or all but one of the phenotypic and morphological characteristics of said variety, optionally as described for each variety in in Table 1 and/or 2, and contains the desired trait and wherein a representative sample of seed of variety NUN 07839 LTL has been deposited under Accession Number NCIMB 42969. In a further embodiment, the desired trait is selected from the group consisting of yield, nutritional value, taste, color, crunchiness, male sterility, herbicide tolerance, insect resistance, pest resistance, disease resistance, environmental stress tolerance, modified carbohydrate metabolism and modified protein metabolism

The invention also provides for progeny of lettuce variety NUN 07839 LTL obtained by further breeding with said variety. In one aspect progeny are F1 progeny obtained by crossing said variety with another plant or S1 progeny obtained by selfing said variety. “Further breeding” encompasses traditional breeding (e.g., selfing, crossing, backcrossing), marker assisted breeding, and/or mutation breeding. In one embodiment, the progeny have one or more (or all) of the distinguishing characteristics when grown under the same environmental conditions. In a further embodiment the progeny have all the physiological and morphological characteristics of said variety when grown under the same environmental conditions. In another embodiment the progeny have one, two, or three distinct traits (qualitative or quantitative) introduced into said variety, while retaining all the other physiological and morphological characteristics of said variety when grown under the same environmental conditions.

The invention also provides a plant having one, two or three physiological and/or morphological characteristics which are different from those of NUN 07839 LTL and which otherwise has all the physiological and morphological characteristics of said variety, wherein a representative sample of seed of variety NUN 07839 LTL has been deposited under Accession Number NCIMB 42969. In particular plants which differ from NUN 07839 LTL in none, one, two or three of the characteristics mentioned in Table 1 and/or 2 are encompassed.

In one aspect, the plant having one, two or three physiological and/or morphological characteristics which are different from those of NUN 07839 LTL and which otherwise has all the physiological and morphological characteristics of NUN 07839 LTL differs from NUN 07839 LTL in one of the distinguishing morphological and/or physiological characteristics selected from: 1) ______; 2) ______; 3) ______; 4) ______; 5) ______; 6) ______; 7) ______; 8) ______; 9) ______; and 10) ______.

In another embodiment the plant having one, two or three physiological and/or morphological characteristics which are different from those of NUN 07839 LTL and which otherwise has all the physiological and morphological characteristics of NUN 07839 LTL differs from NUN 07839 LTL in one, two or three morphological or physiological characteristic other than the 10 “distinguishing morphological and/or physiological characteristics” of NUN 07839 LTL: 1) ______; 2) ______; 3) ______; 4) ______; 5) ______; 6) ______; 7) ______; 8) ______; 9) ______; and 10) ______.

A lettuce according to the invention, such as NUN 07839 LTL, or its progeny, or a plant having all physiological and/or morphological characteristics but one, two or three which are different from those of NUN 07839 LTL, can also be reproduced using vegetative reproduction methods. Therefore, the invention provides for a method of producing a plant, or a part thereof, of variety NUN 07839 LTL, comprising vegetative propagation of said variety. Vegetative propagation comprises regenerating a whole plant from a plant part of variety NUN 07839 LTL (or from a progeny of said variety or from or a plant having all physiological and/or morphological characteristics of said variety but one, two or three different characteristics), such as a cutting, a cell culture or a tissue culture.

The invention also concerns methods of vegetatively propagating a plant of the invention. In certain embodiments, the method comprises the steps of: (a) collecting tissue or cells capable of being propagated from a plant of the invention; (b) cultivating said tissue or cells to obtain proliferated shoots; and (c) rooting said proliferated shoots, to obtain rooted plantlets. Steps (b) and (c) may also be reversed, i.e. first cultivating said tissue to obtain roots and then cultivating the tissue to obtain shoots, thereby obtaining rooted plantlets. The rooted plantlets may then be further grown, to obtain plants. In one embodiment, the method further comprises step (d) growing plants from said rooted plantlets.

The invention also provides for a vegetatively propagated plant of variety NUN 07839 LTL (or from progeny of said variety or from or a plant having all but one, two or three physiological and/or morphological characteristics of NUN 07839 LTL, or a part thereof, having one or more distinguishing characteristics and/or all or all but one, two or three of the morphological and physiological characteristics of NUN 07839 LTL when grown under the same environmental conditions.

In one aspect a haploid plant and/or a doubled haploid plant of NUN 07839 LTL, or a plant having all but one, two or three physiological and/or morphological characteristics of NUN 07839 LTL, or progeny of any of these, are encompassed herein. Haploid and doubled haploid (DH) plants can, for example, be produced by cell or tissue culture and chromosome doubling agents and regeneration into a whole plant. For DH production chromosome doubling may be induced using known methods, such as colchicine treatment or the like.

A part of a variety of the invention, i.e. NUN 07839 LTL (or of progeny of said variety or of a plant having all physiological and/or morphological characteristics but one, two or three which are different from those of said variety) encompasses any cells, tissues, organs obtainable from the seedlings or plants, such as but not limited to: a lettuce head or a part thereof, a leaf or a part thereof, a cutting, hypocotyl, cotyledon, seedcoat, pollen and the like. Such parts can be stored and/or processed further.

Encompassed are therefore also food or feed products comprising one or more of such parts, such as chopped, sliced, cut, ripped, bagged, preserved, cooked or frozen lettuce heads or leaves from NUN 07839 LTL or from progeny of said variety, such as a plant having all but one, two or three physiological and/or morphological characteristics of NUN 07839 LTL. Such a food or feed product comprises or consists of a plant part described herein wherein the plant part can be identified as a part of the plant of the invention NUN 07839 LTL. Preferably, said plant part is a lettuce head or part thereof or a leaf or a part thereof or an extract from a lettuce head or another plant part described herein.

For example, containers such as cans, boxes, crates, bags, cartons, Modified Atmosphere Packagings, films (e.g. biodegradable films), etc. comprising a plant or a parts of a plant (fresh and/or processed, preferably comprising a head or a leaf) described herein or a seed of NUN 07839 LTL are also provided herein. Marketable lettuce heads or leaves are generally sorted by size and quality after harvest. Alternatively the lettuce heads or leaves can be sorted by leaf size, shape, texture, glossiness or color.

Also provided are plant parts obtainable from variety NUN 07839 LTL (or from progeny of said variety or from a plant having all but one, two or three physiological and/or morphological characteristics which are different from those of NUN 07839 LTL or from a vegetatively propagated plant of NUN 07839 LTL (or from its progeny or from a plant having all but one, two or three physiological and/or morphological characteristics which are different from those of NUN 07839 LTL), being selected from the group consisting of a head, a harvested head, a part of a head, a leaf, a part of a leaf, pollen, an ovule, a cell, a petiole, a shoot or a part thereof, a stem or a part thereof, a root or a part thereof, a root tip, a cutting, a core, a seed, a part of a seed, seedcoat or another maternal tissue which is part of a seed grown on NUN 07839 LTL.

In one embodiment, the invention provides for extracts of a plant described herein and compositions comprising or consisting of such extracts. In a preferred embodiment, the extract consists of or comprises tissue of a plant described herein or is obtained from such tissue.

In still yet another aspect, the invention provides a method of determining the genotype of a plant of the invention comprising the step of detecting in the genome (e.g., a sample of nucleic acids) of the plant at least a first polymorphism or an allele. The skilled person is familiar with many suitable methods of genotyping, detecting a polymorphism or detecting an allele including restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, and hybridization to DNA microarrays or beads. Alternatively, the entire genome could be sequenced. The method may, in certain embodiments, comprise detecting a plurality of polymorphisms in the genome of the plant, for example by obtaining a sample of nucleic acid from a plant and detecting in said nucleic acids a plurality of polymorphisms. The method may further comprise storing the results of the step of detecting the plurality of polymorphisms on a computer readable medium.

All documents (e.g., patent publications) are herein incorporated by reference in their entirety. Cited references:

“USDA descriptors” are the plant variety descriptors described for lettuce in the “Objective description of Variety—Lettuce (Lactuca sativa L.)”, as published by U.S. Department of Agriculture, Agricultural Marketing Service, Science and Technology, Plant Variety Protection Office, Beltsville, Md. 20705 and which can be downloaded from the world wide web at ams.usda.gov/ under sites/default/files/media/01-Lettuce%20ST-470-01%202015.pdf.
“UPOV descriptors” are the plant variety descriptors described for lettuce in the “Guidelines for the Conduct of Tests for Distinctness, Uniformity and Stability,” TG/013/10 (Geneva 2006, last updated Mar. 20, 2013), as published by UPOV (International Union for the Protection of New Varieties and Plants) and which can be downloaded from the world wide web at upov.int/ under edocs/tgdocs/en/tg013.pdf.

### Teng et al., HortScience. 1992, 27(9): 1030-1032

### Teng et al., HortScience. 1993, 28(6): 669-1671,

### Zhang et al., Journal of Genetics and Breeding. 1992, 46(3): 287-290).

Clewer, A. G., and D. H. Scarisbrick. 2001 contains a comprehensive explanation of the whole comparison process).
Gonai et al., J. of Exp. Bot., 55(394): 111, 2004

### Louise Jackson et al, Publication 7215 ISBN 978-1-60107-007-4

### Louise Jackson et al, Publication 7216 ISBN 978-1-60107-008-1

### US 2008/0222949

### EP 1 197 137 A1

The Munsell Book of Color (Munsell Color Macbeth Division of Kollmorgan Instruments Corporation) or using the Royal Horticultural Society Chart (World wide web at rhs.org.uk/Plants/RHS-Publications/RHS-colour-charts).

### WO2013182646

### Brotman et al., Theor Appl Genet (2002) 104:1055-1063

Allard, 1960, John Wiley & Sons, Inc.: Principles of plant breeding: 119-128, Library of Congress Catalog Card Number: 60-14240.

### Principles of Plant Genetics and Breeding, 2007, George Acquaah, Blackwell Publishing, ISBN-13: 978-1-4051-3646-4.

### Dziechciarková et al, PLANT SOIL ENVIRON., 50, 2004 (2): 47-58).

### EXAMPLES

### Development of NUN 07839 LTL

The inbred variety NUN 07839 LTL was developed from an initial cross between lettuce lines. The female and male parents were crossed to produce seeds. After the cross, progeny were self-pollinated or backcrossed, followed by pedigree selection and line selection. NUN 07839 LTL can be propagated by seeds or vegetatively, or by regeneration of a tissue culture. The seeds of NUN 07839 LTL can be grown to produce inbred plants and parts thereof (e.g. lettuce heads and leaves).

The variety is uniform and genetically stable. This has been established through evaluation of horticultural characteristics. Several seed production events resulted in no observable deviation in genetic stability. The Applicant concluded that NUN 07839 LTL is uniform and stable.

### Deposit Information

A total of 2500 seeds of variety NUN 07839 LTL were deposited according to the Budapest Treaty by Nunhems B.V. on Feb. 13, 2018, at the NCIMB Ltd., Ferguson Building, Craibstone Estate, Bucksburn, Aberdeen AB21 9YA, United Kingdom (NCIMB). The deposit has been assigned NCIMB 42969. A deposit of NUN 07839 LTL is also maintained at Nunhems B.V.

Access to the deposits will be available during the pendency of this application to persons determined by the Director of the U.S. Patent Office to be entitled thereto upon request. Subject to 37 C.F.R. § 1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

The most similar variety to NUN 07839 LTL is referred to as REFERENCE VARIETY, a variety from ______ with the commercial name ______.

In Table 1 and 2 a comparison between NUN 07839 LTL and its REFERENCE VARIETY is shown based on a trial in the USA. Trial location ______, transplanting date: ______, harvesting date: ______.

Two replications of 50 plants of each variety, from which 15 plants or plant parts were randomly selected, were used to measure characteristics. For numerical characteristics averages were calculated. For non-numerical characteristics the type/degree was determined. In Table 1 the USDA descriptors of NUN 07839 LTL (this application) and REFERENCE VARIETY (commercial variety) are listed.

In accordance with one aspect of the present invention, there is provided a plant having the physiological and morphological characteristics of NUN 07839 LTL as presented in Table 1 and 2.

TABLE 1
Objective description of variety NUN 07839 LTL and their
REFERENCE VARIETY.

| | NUN | REFERENCE |
|---|---|---|
| | 07839 | VARIETY |
| USDA descriptor | LTL | — |
| Plant type | [symbol] | [symbol] |
| 1 = Cutting/Leaf; 02 = Butterhead; 03 = Bibb; | | |
| 04 = Cos or Romaine; 05 = Great Lakes Group; | | |
| 06 = Vanguard Group; 07 = Salinas Group; | | |
| 08 = Eastern (Ithaca) Group; 09 = Stem; 10 = | | |
| Latin; 11 = Other (Specify) | | |
| Seed | | |
| Color: 1 = White (Silver Gray); 2 = Black (Grey | | |
| Brown) | | |
| Light dormancy: 1 = light required; 2 = light not | | |
| required | | |
| Heat dormancy: 1 = susceptible; 2 = not | | |
| susceptible | | |
| Cotyledon to fourth leaf stage | | |
| Shape of Cotyledons: 1 = broad, 2 = | | |
| intermediate, 3 = spatulate | | |
| Shape of fourth leaf: 1= Transverse oval ; 2 = | | |
| Round; 3 = oval; 4 = Elongated; 5 = Lanceolate | | |
| 6 = pinnately lobed | | |
| LENGTH/WIDTH INDEX OF FOURTH LEAF: | | |
| L/W × 10 | | |
| APICAL MARGIN: 1 = Entire; 2 = | | |
| Crenate/Gnawed; 3 = Finely Dentate; 4 = | | |
| Moderately Dentate; 5 = Coarsely Dentate; 6 = | | |
| Incised; 7 = Lobed; 8 = Other (Specify) _ | | |
| BASAL MARGIN: | | |
| UNDULATION: 1 = Flat 2 = Slight 3 = Medium | | |
| 4 = Marked | | |
| Green color: 1 = very light green, 2 = light green, | | |
| 3 = medium green, 4 = dark green; 5 = Very | | |
| Dark Green; 6 = other | | |
| Mature leaves (harvest mature outer leaves): | | |
| Anthocyanin: | | |
| Distribution: 1 = absent; 2 = Margin Only (Big | | |
| Boston); 3 = spotted (California Cream Butter); | | |
| 4 = throughout (Prize Head); 5 = Other (Specify) | | |
| Concentration: 1 = light, 2 = moderate, 3 = | | |
| intense | | |
| CUPPING: 1 = Uncupped 2 = Slight 3 = | | |
| Markedly | | |
| REFLEXING: 1 = None 2 = Apical Margin 3 = | | |
| Lateral Margins | | |
| Margin: | | |
| Incision depth (deepest penetration of the | | |
| margin): 1 = absent/shallow (Dark Green | | |
| Boston), 2 = moderate (Vanguard), 3 = deep | | |
| (Great Lakes 659) | | |
| Incision density: 3 = sparse, 5 = medium, 7 = | | |
| dense, 9 = very dense | | |
| Indentation (finest divisions of the margin): 1 = | | |
| entire, 2 = shallowly dentate (Great Lake 65), 3 = | | |
| deeply dentate (Great Lake 659); 4 = Crenate | | |
| (Vanguard); 5 = Other (Specify) | | |
| Undulations of the apical margin: 1 = | | |
| absent/slight (Dark Green Boston), 2 = moderate | | |
| (Vanguard), 3 = strong (Great Lakes 659) | | |
| Green color: 1 = very light green, 2 = light green, | | |
| 3 = medium green, 4 = dark green; 5 = Very | | |
| Dark Green; 6 = other | | |
| Anthocyanin: | | |
| Distribution: 1 = absent; 2 = Margin Only (Big | | |
| Boston); 3 = spotted (California Cream Butter); | | |
| 4 = throughout (Prize Head); 5 = Other (Specify) | | |
| Concentration: 1 = light, 2 = moderate, 3 = | | |
| intense | | |
| Size: 1 = small, 2 = medium, 3 = large | | |
| Glossiness: 1 = dull, 2 = moderate, 3 = glossy | | |
| Blistering: 1 = absent/slight, 2 = moderate, 3 = | | |
| strong | | |
| Leaf thickness: 1 = thin, 2 = intermediate, 3 = | | |
| thick | | |
| Trichomes; 1 = absent, 2 = present | | |
| Plant | | |
| Spread of frame leaves (cm) | | |
| Head diameter (market trimmed with single cap | | |
| leaf) | | |
| Head shape: 1 = flattened, 2 = Slightly Flattened; | | |
| 3 = Spherical; 4 = elongate, 5 = non-heading; 6 = | | |
| nonheading | | |
| Head size class: 1 = small, 2 = medium, 3 = large | | |
| Head per carton | | |
| Head weight (gram) | | |
| Head firmness: 1 = loose, 2 = Moderate; 3 = | | |
| Firm, 4 = very firm | | |
| Butt | | |
| Shape | | |
| 1 = slightly concave, 2 = flat, 3 = rounded; 4 = | | |
| V-shaped | | |
| Midrib | | |
| 1 = Flattened, 2 = Moderately Raised, | | |
| 3 = prominently raised | | |
| Core | | |
| Diameter at base of head (mm) | | |
| Ratio of head spread frame leaves/core diameter | | |
| Core height from base of head to apex( mm) | | |
| range | | |
| Bolting (first water date:) | | |
| Bolting class: 1 = very slow, 2 = slow, 3 = | | |
| medium, 4 = rapid, 5 = very rapid | | |
| Maturity (earliness of harvest-mature head | | |
| formation) | | |
| Spring (days) | | |
| Adaptation: | | |
| Primary regions of adaptation | | |
| Season : 0 = not tested, 1 = not adapted, 2 = | | |
| adapted | | |
| Greenhouse: 0 = not tested, 1 = not adapted, 2 = | | |
| adapted | | |
| Soil type: 1 = mineral, 2 = organic, 3 = both | | |
| Viral Diseases: | | |
| Fungal/bacterial diseases: | | |
| Insects: (not tested) | | |
| Physiological stresses: | | |
| Post Harvest stress | | |

[symbol]

indicates data missing or illegible when filed

TABLE 2

| | | REFERENCE |
|---|---|---|
| | | VARIETY |
| Non-USDA descriptor | NUN 07839 LTL | — |
| [symbol] | | |

[symbol]

indicates data missing or illegible when filed

Table 1 and 2 contain typical values. Values may vary due to environment. Other values that are substantially equivalent are also within the scope of the invention. N.A.=not applicable; n.r.=not recorded.

1. A plant, plant part or seed of lettuce variety NUN 07839 LTL, wherein a representative sample of said seed has been deposited under Accession Number NCIMB 42969.

2. The plant part of claim 1, further defined as a head, a leaf, pollen, an ovule, a fruit, a scion, a rootstock, cutting, flower or a part of any of these or a cell.

3. A seed from which the plant of claim 1 can be grown.

4. A seed grown on the plant of claim 1.

5. A lettuce plant, or a part thereof which does not significantly differ from the plant of claim 1 in any of the distinguishing characteristics selected from the group consisting of: 1) ______; 2) ______; 3) ______; 4) ______; 5) ______; 6) ______; 7) ______; 8) ______; 9) ______; and 10) ______.

6. A lettuce plant, or a part thereof which does not differ from the plant of claim 1 when grown under the same conditions, determined at the 5% significance level.

7. A tissue or cell culture of regenerable cells of the plant of claim 1.

8. The tissue or cell culture according to claim 7, comprising cells or protoplasts from a plant part selected from the group consisting of embryos, meristems, cotyledons, hypocotyl, pollen, leaves, anthers, roots, root tips, pistil, petiole, flower, fruit, seed, stem and stalks.

9. A lettuce plant regenerated from the tissue or cell culture of claim 7, wherein the plant has all or all but one of the physiological and morphological characteristics of the plant of NUN 07839 LTL, wherein a representative sample of seed of said variety has been deposited under Accession Number NCIMB 42969, when determined at the 5% significance level when grown under the same conditions.

10. A method of producing of the plant of claim 1, or a part thereof, comprising vegetative propagation of the plant of NUN 07839 LTL, wherein a representative sample of seed of said variety has been deposited under Accession Number NCIMB 42969.

11. The method of claim 10, wherein said vegetative propagation comprises regenerating a whole plant from a part of the plant of claim 1.

12. The method of claim 10, wherein said part is a cutting, a cell culture or a tissue culture.

13. A vegetative propagated plant, or a part of said propagated plant, propagated from a plant or plant part of claim 1 wherein the plant has all or all but one of the physiological and morphological characteristics of NUN 07839 LTL, wherein a representative sample of said seed has been deposited under Accession Number NCIMB 42969, wherein said characteristics are determined at the 5% significance level for plants grown under the same conditions.

14. A method of producing a lettuce plant, comprising crossing the plant of claim 1 with a second lettuce plant one or more times, and selecting progeny from said crossing and optionally allowing the progeny to form seed.

15. A first generation progeny of plant of claim 1.

16. A lettuce plant having one, two or three physiological and/or morphological characteristics which are different from those of the plant of claim 1 and which otherwise has all the physiological and morphological characteristics of the plant of claim 1 as listed in Table 1 and/or 2, when grown under the same conditions and determined at the 5% significance level.

17. A food or feed product comprising the plant part of claim 2.

18. A lettuce plant comprising at least a first set of the chromosomes of the plant of claim 1.

19. The plant of claim 1 further comprising a single locus conversion, wherein said plant has all or all but one, two or three of the morphological and physiological characteristics of the plant of claim 1 when grown under the same conditions , optionally wherein the single locus conversion confers a trait selected from the group consisting yield, storage properties, color, male sterility, herbicide tolerance, insect resistance, pest resistance, disease resistance, environmental stress tolerance, modified carbohydrate metabolism and modified protein metabolism.

20. A method of producing the plant of claim 1 further comprising a single locus conversion, comprising steps:
a. crossing the plant of claim 1 with a second plant comprising a single locus and obtaining progeny of said crossing; wherein the single locus in the second plant is the locus to be introduced into the plant of claim 1;
b. optionally selfing the progeny of step a and obtaining progeny of said selfing.
c. optionally crossing the progeny plant with the plant of claim 1 and obtaining progeny of said cross;
d. optionally repeating step c one or more times in succession with the progeny obtained in step c

21. A plant obtained by the method of claim 20.

22. A plant or a cell thereof produced in a method of producing a plant having a desired trait, wherein the method comprises mutating or transforming a lettuce plant of variety NUN 07839 LTL and selecting a plant with the desired trait, wherein the mutated plant retains all or all but one of the phenotypic and morphological characteristics of variety NUN 07839 LTL as described in Table 1 and/or 2 and contains the desired trait and wherein a representative sample of seed of variety said variety has been deposited under Accession Number NCIMB 42969.

23. The plant or cell of claim 22, wherein the desired trait is selected from the group consisting of yield, storage properties, color, male sterility, herbicide tolerance, insect resistance, pest resistance, disease resistance, environmental stress tolerance, modified carbohydrate metabolism and modified protein metabolism.

24. A container comprising a plant, plant part or seed of claim 1.
